(19) United States
(12) Patent Application Publication
(10) Pub. No.: US 2026/0334757 A1
(43) Pub. Date: Oct. 29, 2026

(54) VISUAL INDICATION OF DISTANCE BETWEEN A DISTAL END OF AN OPTICAL FIBER AND A TARGET DURING LASER LITHOTRIPSY

(71) Applicant: Lumenis Ltd., Yokneam Ilit (IL)

(72) Inventors: Hiba Zarad, Haifa (IL); Tomer Gartenhaus, Yad Hana (IL); Saman Naser, Tira (IL); Amir Finkel, Zichron Yaakov (IL)

(73) Assignee: Lumenis Ltd., Yokneam Ilit (IL)

(21) Appl. No.: 19/653,522

(22) Filed: Apr. 21, 2026

Related U.S. Application Data
(60) Provisional application No. 63/796,010, filed on Apr. 28, 2025.

(51) Int. Cl. A61B 1/00; A61B 1/018; A61B 18/26; A61B 18/00; A61B 90/00

(52) U.S. Cl. CPC A61B 1/0005; A61B 1/00006; A61B 1/0004; A61B 1/018; A61B 18/26; A61B 2018/00982; A61B 2090/061

(57) ABSTRACT

Described herein are laser emitting medical devices and techniques implementable by laser emitting medical devices to determine and display a calculated distance between a distal end of an optical fiber and a target and to display a qualitative indication of the distance on a graphical user interface (GUI) associated with an endoscopic view of the target.

### PRIORITY

This application claims the benefit of priority under 35 USC § 119 to United States Provisional Patent Application Serial No. 63/796,010, filed Apr. 28, 2025, which is incorporated by reference herein in its entirety and for all purposes.

### TECHNICAL FIELD

The present disclosure generally relates to surgical laser system. Particularly, but not exclusively, the present disclosure relates to visually indicating the distance between a target and a distal end of an optical fiber during a laser lithotripsy procedure.

### BACKGROUND

Medical lasers are used in a variety of procedures. Among a number of such procedures, laser energy is directed towards a target using an optical fiber as a conduit for the laser energy. One such procedure, to address renal calculi (e.g., kidney stones) is ureteral endoscopy, or lithotripsy. A typical lithotripsy procedure involves inserting an endoscopic (e.g., ureteroscope, or the like) into the urinary tract of a patient. A camera of the endoscope is used to locate the calculi for removal. As part of the removal process, an optical fiber is inserted into the treatment area via a working channel of the endoscope. Laser energy is generated by a surgical laser console and the target is illuminated and/or irradiated with the laser energy via the optical fiber. The laser energy operates to disintegrate, dust, ablate, fragment and/or destroy the calculi.

As will be appreciated by those of ordinary skill in the art, the laser energy is effective up to a specific distance. Thus, there is a need to identify the distance between the distal end of the optical fiber and the target (e.g., urinary stone) and communicate this distance to the physician.

### SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

The present disclosure provides a surgical laser console configured to determine a distance between a distal end of an optical fiber and a target (e.g., renal calculi, which can be referred to as a urinary stone or just a stone). Further, the surgical laser console is configured to visually display the distance for a physician (e.g., via a graphical user interface, or the like). In some embodiments, the surgical laser console can be configured to communicate the distance to another console, such as, an endoscopic console, where a visual indication of the distance can be displayed by the other console.

In some embodiments, the disclosure can be implemented as a computer implemented method. The method can comprise generating, by a processor during a surgical procedure, a visual representation of a distance between a distal end of an optical fiber and a target, wherein the target is disposed in a treatment environment and wherein the optical fiber is inserted into the treatment environment via a working channel of an endoscope; and sending, by the processor, control signals to a display to cause the display to display the visual representation during the surgical procedure.

With further embodiments, the method can comprise receiving, by the processor from a surgical laser console, an indication of the distance.

With further embodiments, the method can comprise receiving, by the processor from an endoscope console during the surgical procedure, indications of an image of the target.

With further embodiments of the method, the control signals cause the display to display the visual representation and the image of the target during the surgical procedure.

With further embodiments, the method can comprise generating, by the processor, a graphical user interface (GUI) comprising the visual representation and the image of the target, wherein the control signals cause the display to display the GUI during the surgical procedure.

With further embodiments, the method can comprise receiving, by the processor from an endoscope during the surgical procedure, indications of an image of the target.

With further embodiments of the method, the control signals cause the display to display the visual representation and the image of the target during the surgical procedure.

With further embodiments, the method can comprise generating, by the processor, a graphical user interface (GUI) comprising the visual representation, wherein the control signals cause the display to display the GUI during the surgical procedure.

With further embodiments, the method can comprise determining the distance by the processor during the surgical procedure.

With further embodiments of the method, the visual representation comprises one or more geometric shapes, one or more numbers, and/or one or more words.

With further embodiments, the method can comprise determining, by the processor during the surgical procedure, whether the distance is less than a threshold value, wherein the visual representation is a first visual representation comprising a quantitative indication that the distance is less than the threshold value based on a determination that the distance is less than the threshold value, or wherein the visual representation is a second visual representation comprising a quantitative indication that the distance is greater than or equal to the threshold value based on a determination that the distance is not less than the threshold value.

With further embodiments of the method, the first visual representation comprises a solid circle and wherein the second visual representation comprises a hollow circle.

In some embodiments, the disclosure can be implemented as a multi-system viewer. The multi-system viewer can comprise a processor; a display coupled to the processor; and memory coupled to the processor and comprising instructions, which when executed by the processor cause the multi-system viewer to execute any of the methods disclosed herein.

In some embodiments, the disclosure can be implemented as an endoscope console. The endoscope console can comprise a processor; a display coupled to the processor; and memory coupled to the processor and comprising instructions, which when executed by the processor cause the endoscope console to execute any of the methods disclosed herein.

In some embodiments, the disclosure can be implemented as a surgical laser console. The surgical laser console can comprise a processor; a display coupled to the processor; and memory coupled to the processor and comprising instructions, which when executed by the processor cause the surgical laser console to execute any of the methods disclosed herein.

In some embodiments, the disclosure can be implemented as a multi-system viewer. The multi-system viewer can comprise a processor; a display coupled to the processor; and memory coupled to the processor and comprising instructions, which instructions when executed by the processor cause the multi-system viewer to generate, during a surgical procedure, a visual representation of a distance between a distal end of an optical fiber and a target, and send, during the surgical procedure, control signals to the display to cause the display to display the visual representation, wherein the target is disposed in a treatment environment and wherein the optical fiber is inserted into the treatment environment via a working channel of an endoscope.

With further embodiments of the multi-system viewer, the instructions when executed by the processor further cause the multi-system viewer to receive, during the surgical procedure, an indication of the distance from a surgical laser console.

With further embodiments of the multi-system viewer, the instructions when executed by the processor further cause the multi-system viewer to receive, during the surgical procedure, an indication of the distance from an endoscope console.

With further embodiments of the multi-system viewer, the instructions when executed by the processor further cause the multi-system viewer to receive, during the surgical procedure, indications of an image of the target.

With further embodiments of the multi-system viewer, the instructions when executed by the processor further cause the multi-system viewer to send the control signals to the display to cause the display to display both the visual representation and the image of the target.

With further embodiments of the multi-system viewer, the instructions when executed by the processor further cause the multi-system viewer to generate a graphical user interface (GUI) comprising at least the visual representation, wherein the control signals cause the display to display the GUI during the surgical procedure.

With further embodiments of the multi-system viewer, the visual representation comprises one or more geometric shapes, one or more numbers, and/or one or more words.

With further embodiments of the multi-system viewer, the instructions when executed by the processor further cause the multi-system viewer to determine, during the surgical procedure, whether the distance is less than a threshold value, wherein the visual representation is a first visual representation comprising a quantitative indication that the distance is less than the threshold value based on a determination that the distance is less than the threshold value, or wherein the visual representation is a second visual representation comprising a quantitative indication that the distance is greater than or equal to the threshold value based on a determination that the distance is not less than the threshold value.

With further embodiments of the multi-system viewer, the first visual representation comprises a solid circle and wherein the second visual representation comprises a hollow circle.

In some embodiments, the disclosure can be implemented as an endoscope console. The endoscope console can comprise a processor; a network interconnect coupled to the processor; a display coupled to the processor; and memory coupled to the processor and comprising instructions, which when executed by the processor cause the endoscope console to receive, from a surgical laser console via the network interconnect during a surgical procedure, an indication of a distance between a distal end of an optical fiber and a target, generate, during the surgical procedure, a visual representation of the distance, and send, during the surgical procedure, control signals to the display to cause the display to display the visual representation, wherein the target is disposed in a treatment environment and wherein the optical fiber is inserted into the treatment environment via a working channel of an endoscope.

With further embodiments of the endoscope console, the instructions when executed by the processor further cause the endoscope console to receive, during the surgical procedure, indications of an image of the target.

With further embodiments of the endoscope console, the instructions when executed by the processor further cause the endoscope console to send the control signals to the display to cause the display to display both the visual representation and the image of the target.

With further embodiments of the endoscope console, the visual representation comprises one or more geometric shapes, one or more numbers, and/or one or more words.

With further embodiments of the endoscope console, the instructions when executed by the processor further cause the endoscope console to determine, during the surgical procedure, whether the distance is less than a threshold value, wherein the visual representation is a first visual representation comprising a quantitative indication that the distance is less than the threshold value based on a determination that the distance is less than the threshold value, or wherein the visual representation is a second visual representation comprising a quantitative indication that the distance is greater than or equal to the threshold value based on a determination that the distance is not less than the threshold value.

With further embodiments of the endoscope console, the first visual representation comprises a solid circle and wherein the second visual representation comprises a hollow circle.

In some embodiments, the disclosure can be implemented as a surgical laser console. The surgical laser console can comprise a network interconnect; a processor; and memory coupled to the processor and comprising instructions, which when executed by the processor cause the surgical laser console to determine, during a surgical procedure, a distance between a distal end of an optical fiber and a target, generate, during the surgical procedure, a visual representation of the distance, and send, to an endoscope console via the network interconnect during the surgical procedure, an indication of the visual representation and control signals to cause a display of the endoscope console to display the visual representation as part of a graphical user interface (GUI), wherein the target is disposed in a treatment environment and wherein the optical fiber is inserted into the treatment environment via a working channel of an endoscope.

With further embodiments of the surgical laser console, the GUI comprises both the visual representation and an image of the target.

With further embodiments of the surgical laser console, the visual representation comprises one or more geometric shapes, one or more numbers, and/or one or more words.

With further embodiments of the surgical laser console, the instructions when executed by the processor further cause the surgical laser console to determine, during the surgical procedure, whether the distance is less than a threshold value, wherein the visual representation is a first visual representation comprising a quantitative indication that the distance is less than the threshold value based on a determination that the distance is less than the threshold value, or wherein the visual representation is a second visual representation comprising a quantitative indication that the distance is greater than or equal to the threshold value based on a determination that the distance is not less than the threshold value.

With further embodiments of the surgical laser console, the first visual representation comprises a solid circle and wherein the second visual representation comprises a hollow circle.

### DESCRIPTION OF THE FIGURES

To easily identify the discussion of any element or act, the most significant digit or digits in a reference number refer to the figure number in which that element is first introduced.

FIG. 1A and FIG. 1B illustrate a first example surgical laser system, in accordance with at least one embodiment.

FIG. 2 illustrates a second example surgical laser system, in accordance with at least one embodiment.

FIG. 3A and 3B illustrate a third example surgical laser system, in accordance with at least one embodiment.

FIG. 4 illustrates a fourth example surgical laser system, in accordance with at least one embodiment.

FIG. 5 illustrates an example interrogation sub-system, in accordance with at least one embodiment.

FIG. 6 illustrates a first method for visually depicting a qualitative indication of a distance between a distal end of an optical fiber and a target, in accordance with at least one embodiment.

FIGS. 7A, 7B, 7C, and 7D illustrate example visual representations of a distance between a distal end of an optical fiber and a target, in accordance with at least one embodiment.

FIG. 8 illustrates a second method for visually depicting a qualitative indication of a distance between a distal end of an optical fiber and a target, in accordance with at least one embodiment.

FIG. 9 illustrates a first example graphical user interface depicting visual representations of a distance between a distal end of an optical fiber and a target, in accordance with at least one embodiment.

FIG. 10 illustrates a second example graphical user interface depicting visual representations of a distance between a distal end of an optical fiber and a target, in accordance with at least one embodiment.

FIG. 11 illustrates a third example graphical user interface depicting visual representations of a distance between a distal end of an optical fiber and a target, in accordance with at least one embodiment.

FIG. 12 illustrates a fourth example graphical user interface depicting visual representations of a distance between a distal end of an optical fiber and a target, in accordance with at least one embodiment.

FIG. 13 illustrates computer executable instructions in accordance with one embodiment.

FIG. 14 illustrates a block diagram of a computing environment.

### DETAILED DESCRIPTION

The foregoing has broadly outlined the features and technical advantages of the present disclosure such that the following detailed description of the disclosure may be better understood. It is to be appreciated by those skilled in the art that the embodiments disclosed may be readily utilized as a basis for modifying or designing other structures for carrying out the same purposes of the present disclosure. The novel features of the disclosure, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

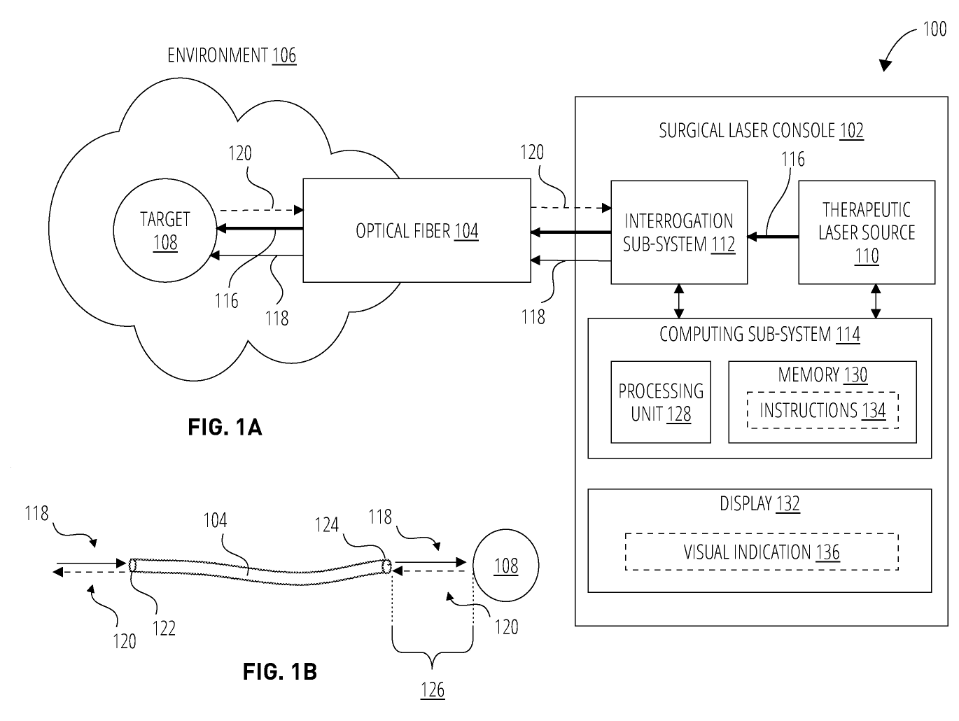

FIG. 1A shows an exemplary surgical laser system 100. In general, the surgical laser system 100 can be configured to determine a distance between a distal end of an optical fiber and a stone (e.g., as part of a laser lithotripsy procedure, or the like).

With some embodiments, the surgical laser system 100 comprises a surgical laser console 102 and an optical fiber 104 coupled to the surgical laser console 102. During a lithotripsy procedure, the optical fiber 104 can be inserted into an environment 106 in which a target 108 is to be treated. In an exemplary lithotripsy procedure, the target 108 is a stone (or renal calculi). However, it will be appreciated that the surgical laser console 102 and optical fiber 104 could be used to treat other types of targets (e.g., tissue, a tumor, a cyst, or the like). With illustrative examples, the target 108 can be disposed in a liquid environment within a subject (e.g., a human, an animal, or the like).

The surgical laser console 102 further includes a therapeutic laser source 110, interrogation sub-system 112, and computing sub-system 114. During operation, the therapeutic laser source 110 is configured to generate therapeutic laser beam 116 while interrogation sub-system 112 is configured to generate interrogation laser beam 118.

In general, the therapeutic laser beam 116 can be configured to “treat” the target 108. For example, the therapeutic laser source 110 can generate therapeutic laser beam 116 having characteristics (e.g., energy, power, pulse width, pulse duration, etc.) configured to fragment, dust, or otherwise destroy the target 108. Accordingly, during operation, the therapeutic laser beam 116 can be generated and transmitted to the target via the optical fiber 104.

Further, generally speaking, the interrogation laser beam 118 is configured to be reflected from the target 108 such that a distance in which the interrogation laser beam 118 is transmitted through the environment 106 can be determined.

In general, the therapeutic laser source 110 can be any of a variety of laser light sources (e.g., solid-state lasers, gas lasers, diode lasers, and fiber lasers). As a specific example, the therapeutic laser source 110 can be a Holmium (Ho) laser, such as a Ho:YAG laser. In yet another example, the therapeutic laser source 110 can be a Thulium (Tm) fiber laser. An example of the interrogation sub-system 112 is provided in greater detail below. However, in general the interrogation sub-system 112 is configured to generate interrogation laser beam 118, which can comprise multiple one or many laser beams, each having different wavelengths.

It is to be appreciated that the surgical laser console 102, and particularly the therapeutic laser source 110 and the interrogation sub-system 112 can include a variety of optical components besides the laser sources described above. For example, the therapeutic laser source 110 and the interrogation sub-system 112 can include (e.g., in different combinations as may be the case) light sources, polarizers, beam splitters, beam combiners, light detectors, wavelength division multiplexers, collimators, circulators, etc.

It is to be appreciated that the environment 106 often includes a liquid medium. For example, during a lithotripsy procedure, the target 108 may be a stone and the environment 106 can include a mixture of urine, irrigation fluid, and blood. Accordingly, the distal end 128 of the optical fiber 104 will also be disposed in the liquid environment during the procedure. When the therapeutic laser beam 116 is emitted from the optical fiber 104, some of the liquid may be vaporized and form a bubble or bubbles.

As will be appreciated, the therapeutic laser beam 116 can attenuate and/or be absorbed by the liquid medium in the environment. As such, there is often an optimal working distance for the therapeutic laser beam 116. To that end, it can be advantageous to determine the distance with which the therapeutic laser beam 116 will be transmitted in the environment.

During operation, the interrogation laser beam 118 can be generated and transmitted towards the target 108 via the optical fiber 104 to determine a distance between the optical fiber 104 and the target 108. More specifically, the interrogation laser beam 118 can be transmitted into the environment 106 and light reflected from objects in the environment 106 (e.g., the target 108, or the like) can be received by the optical fiber 104 and transmitted back to the surgical laser console 102. For example, responsive to incidence of the interrogation laser beam 118 on the target 108, target reflected light 120 may be reflected by the target 108.

FIG. 1B illustrates an example of the optical fiber 104 receiving the interrogation laser beam 118, transmitting the interrogation laser beam 118 to the target 108, and receiving target reflected light 120.

In general, the optical fiber 104 can be optically coupled to the surgical laser console 102 at a proximal end 122 of the optical fiber 104. As such, the interrogation laser beam 118 can be optically coupled to the optical fiber 104, transmitted through the optical fiber 104, and emitted from a distal end 124 of the optical fiber 104.

This figure depicts the optical fiber 104 “targeting” the target 108, which is disposed a distance 126 away from the distal end 124 of the optical fiber 104. Said differently, the distal end 124 is pointed at the target 108 such that the interrogation laser beam 118 emitted from the distal end 124 is incident on the target 108 after being transmitted the distance 126 through the environment 106. In such an orientation, target reflected light 120 is reflected from the target 108 (e.g., as a result of the interrogation laser beam 118 being incident on the target 108). Further, the target reflected light 120 is received at the distal end 124 of the optical fiber 104, transmitted through the optical fiber 104, and optically coupled to the surgical laser console 102.

Returning to FIG. 1A, the computing sub-system 114 is depicted comprising at least a processing unit 128, memory 130, and display 132. The memory 130 can store instructions 134 while the display can display visual indication 136. With some examples, the computing sub-system 114 may be provided by a computing device (e.g., a laptop, a desktop, a mobile phone, a tablet, or the like). The processing unit 128 can include circuitry arranged to execute the instructions 134 stored in memory 130, which instructions when executed cause the computing sub-system 114 to send control signals to the therapeutic laser source 110 and the interrogation sub-system 112. For example, the processing unit 128 can execute instructions 134 to cause the computing sub-system 114 to send control signals to the interrogation sub-system 112 to cause the interrogation sub-system 112 to generate interrogation laser beam 118 and also to receive signals from the interrogation sub-system 112 comprising indications of target reflected light 120.

Further, the processing unit 128 can execute instructions 134 to cause the computing sub-system 114 to determine the distance 126 between the distal end 124 of the optical fiber 104 and the target based on the interrogation laser beam 118 and the target reflected light 120. This is described in greater detail below. The processing unit 128 can execute instructions 134 to further cause the computing sub-system 114 to send control signals to the therapeutic laser source 110 to cause the therapeutic laser source 110 to generate the therapeutic laser beam 116 as discussed above.

Additionally, the processing unit 128 can execute instructions 134 to further cause the computing sub-system 114 to generate visual indication 136 and cause the display 132 to display the 136. The visual indication 136 can comprise an indication of the distance 126.

With some example lithotripsy procedures, the optical fiber 104 can be inserted into a patient's kidney via an endoscope.

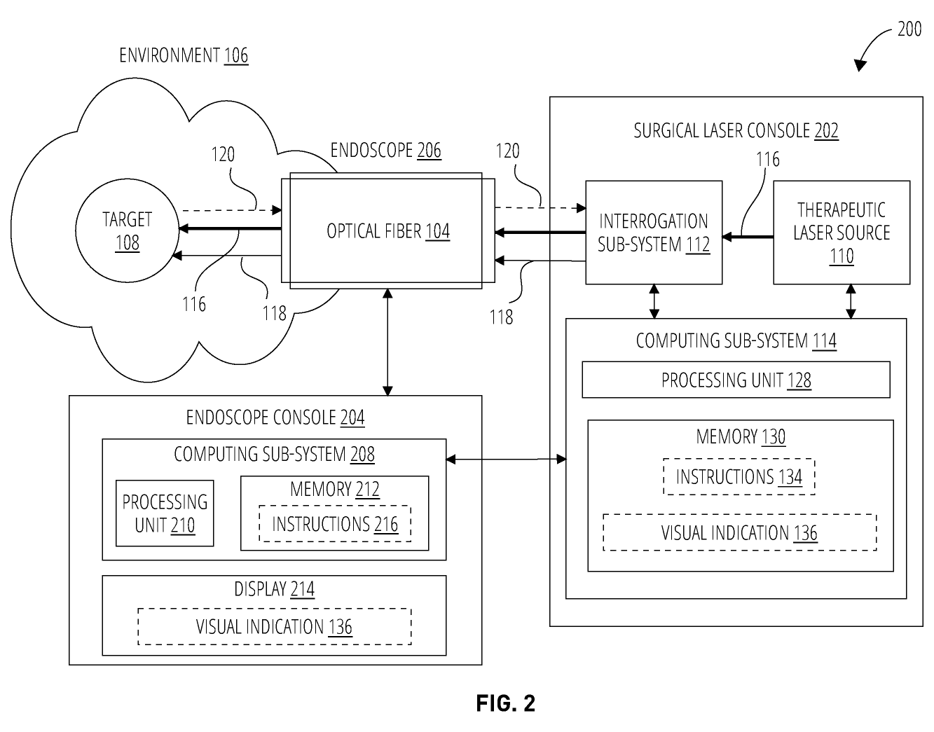

FIG. 2 shows an exemplary surgical laser system 200 comprising a surgical laser console 202, endoscope console 204, endoscope 206, and optical fiber 104. The surgical laser system 200 can be configured like the surgical laser system 100 to determine the distance 126 between the distal end 124 of the optical fiber 104 and the target 108 and to display an indication of the distance on a display. The surgical laser system 200 can include many of the same components as the surgical laser system 100 depicted in FIGS. 1A and 1B. For convenience and brevity, FIG. 2 is described with reference to FIGS. 1A and 1B. However, it is to be appreciated that the surgical laser system 200 can comprise different elements than those described with reference to FIGS. 1A and 1B.

During operation, the endoscope 206 can be inserted into the environment 106. The endoscope console 204 can comprise a computing sub-system 208, which can be like the computing sub-system 114. For example, the computing sub-system 208 can include processing unit 210, memory 212, and display 214. The memory 212 can store instructions 216 while the display can display visual indication 136. With some examples, the computing sub-system 208 may be provided by a computing device (e.g., a laptop, a desktop, a mobile phone, a tablet, or the like). The processing unit 210 can include circuitry arranged to execute the instructions 216 stored in memory 212, which instructions when executed cause the computing sub-system 208 to send control signals to and receive control signals from the endoscope 206. For example, processing unit 210 can execute instructions 216 to receive indications of images captured by a camera (not shown) of the endoscope 206 and display indications of the images on the display 214 such that the endoscope 206 can be navigated to the environment 106 and further that the target can be visualized during a treatment procedure.

With some embodiments, the processing unit 128 can execute instructions 134 to cause the computing sub-system 114 to determine the distance 126 as outlined above and send indications of the distance 126 to the computing sub-system 208 or to generate visual indication 136 of the distance 126 and send the visual indication 136 to the computing sub-system 208.

Further, the processing unit 210 can execute instructions 216 to cause the computing sub-system 208 to receive indications of the distance 126 from the computing sub-system 114 and to generate visual indication 136 comprising indications of the distance 126. In another example, the processing unit 210 can execute instructions 216 to cause the computing sub-system 208 to receive indications of the visual indication 136 from the computing sub-system 114. In either embodiment, the processing unit 210 can execute instructions 216 to cause the display 214 to display the visual indication 136.

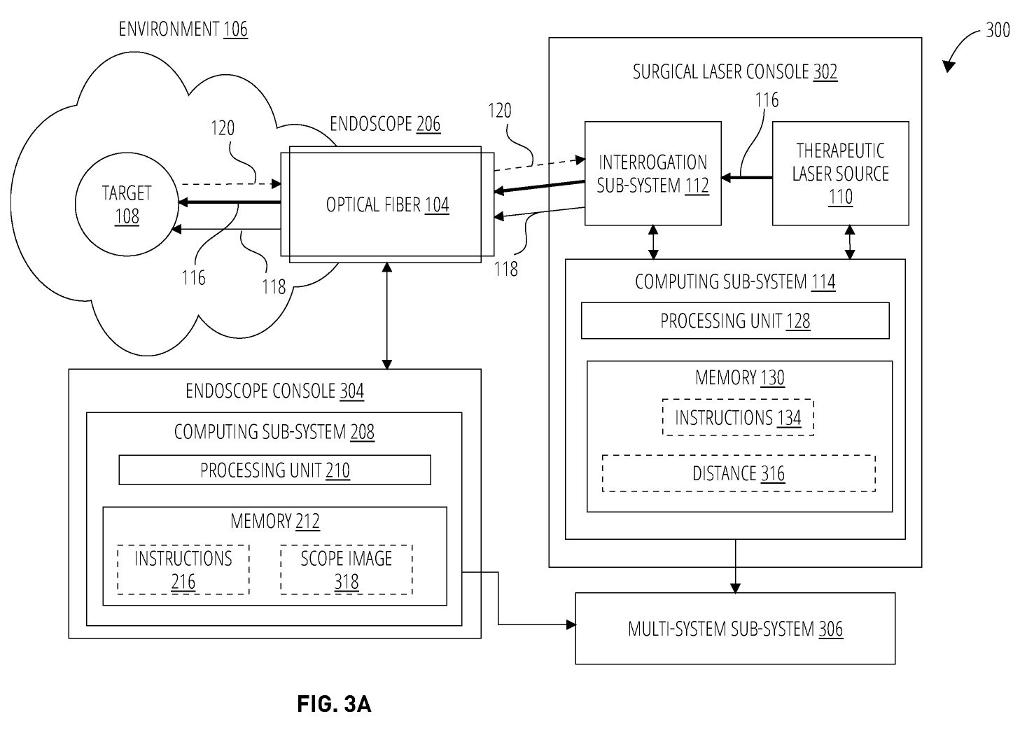

FIGS. 3A and 3B shows an exemplary surgical laser system 300 comprising a surgical laser console 302, an endoscope console 304, a multi-system sub-system 306, the optical fiber 104, and the endoscope 206. The surgical laser system 300 can be configured like the surgical laser system 200 to determine the distance 126 between the distal end 124 of the optical fiber 104 and the target 108 and to display an indication of the distance on a display. The surgical laser system 300 can include many of the same components as the surgical laser system 100 depicted in FIGS. 1A and 1B or the surgical laser system 200 depicted in FIG. 2. For convenience and brevity, FIGS. 3A and 3B are described with reference to these embodiments. However, it is to be appreciated that the surgical laser system 300 can comprise different elements than those described with reference to FIGS. 1A, 1B, and 2.

As outlined above, the endoscope 206 can be inserted into the environment 106 while the optical fiber 104 can be inserted into the environment via a working channel (not shown) of the endoscope 206.

The multi-system sub-system 306 can be provided with a processing unit 308, memory 310, and a display 312. With some examples, the computing multi-system sub-system 306 may be provided by a computing device (e.g., a laptop, a desktop, a mobile phone, a tablet, or the like). The processing unit 308 can include circuitry arranged to execute instructions 314 stored in memory 310, which instructions when executed cause the multi-system sub-system 306 to send control signals to and receive control signals from the surgical laser console 302 and the endoscope console 304.

The surgical laser console 302 can be like the surgical laser console 102 or the surgical laser console 202 and can include the therapeutic laser source 110, the interrogation sub-system 112, and the computing sub-system 114. During operation, the processing unit 128 can execute instructions 134 to cause the computing sub-system 114 to determine the distance 126 as outlined above and send indications of the distance 126 to the multi-system sub-system 306. The endoscope console 304 can comprise the computing sub-system 208. In some embodiments, the display 214 of computing sub-system 208 is omitted and/or external to the endoscope console 304. As noted, the memory 212 can store instructions 216, which when executed by the processing unit 210 cause the endoscope console 304 to receive indications of images captured by the endoscope camera (not shown). In some embodiments, these images can be stored as scope images 318. Further, the processing unit 210 can execute the instructions 216 to send indications of the scope images 318 to the multi-system sub-system 306.

The processing unit 308 can execute instructions 314 to further cause the multi-system sub-system 306 to receive the distance 316 and the scope images 318 and to generate multi-view images 320 comprising visualizations of the distance 126 and the environment 106 and/or the target 108. Additionally, the processing unit 308 can execute instructions 314 to cause the multi-system sub-system 306 to cause display 312 to display the multi-view images 320.

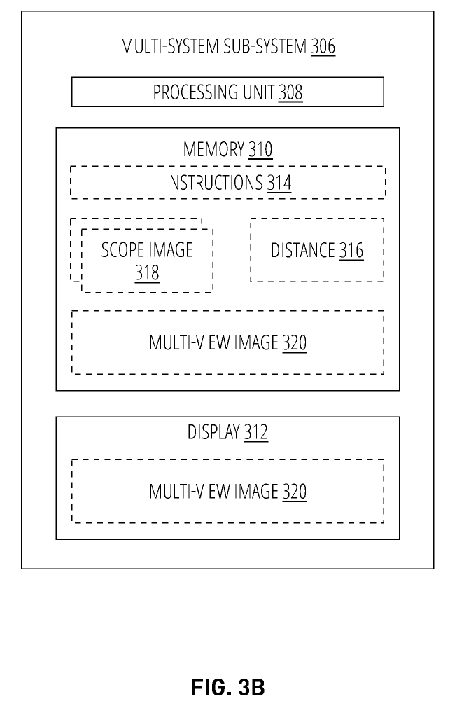

In some embodiments, a surgical laser system like the surgical laser system 100 and/or surgical laser system 200 could be provided with a remote and/or portable display arranged to display the visual indication 136. For example, FIG. 4 illustrates an example surgical laser system 400, which includes the elements of the surgical laser system 100 with the difference being that the display 132 is not provided as part of the surgical laser console 102 and a remote display coupled to the computing sub-system 114 is provided.

During operation, the processing unit 128 can execute instructions 134 to cause the computing sub-system 114 to send indications of the visual indication 136 to the display 402 and cause the display 402 to display the visual indication 136.

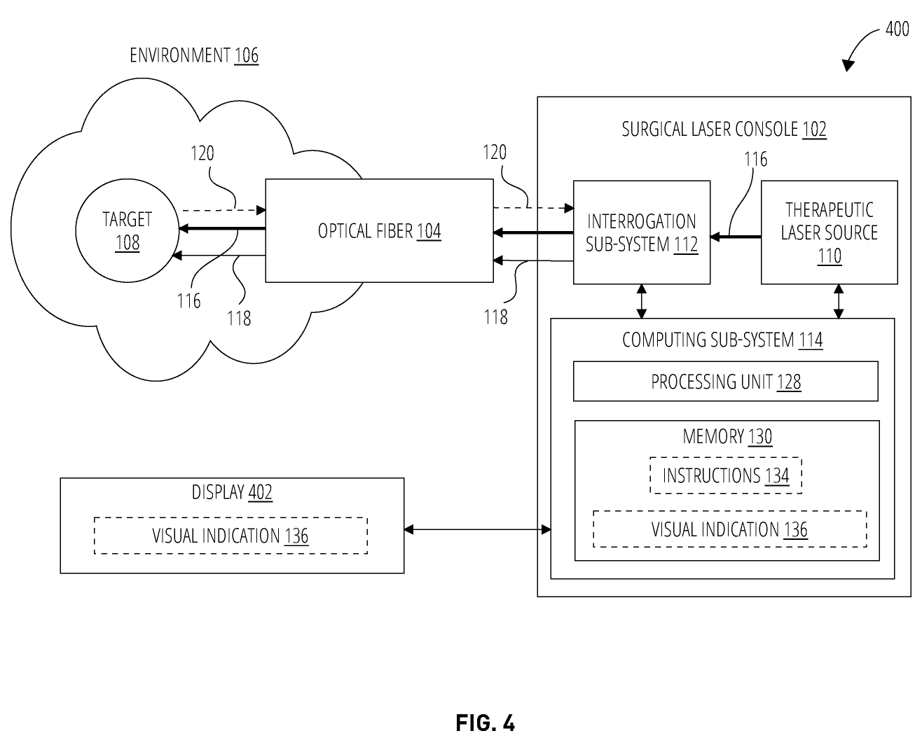

It is noted that determining the distance 126 between the distal end 124 of the optical fiber 104 and the target 108 is beyond the scope of this disclosure. However, for completeness of disclosure, FIG. 5 illustrates an example interrogation sub-system 500, which can be implemented as interrogation sub-system 112 of surgical laser system 100 of FIG. 1A, surgical laser system 200 of FIG. 2, or surgical laser system 400 of FIG. 4. The interrogation sub-system 500 can include an interrogation laser source 502, a beam splitter 504, a reference detector 506, optics 508, and a signal detector 510. The interrogation laser source 502 can be arranged to generate interrogation laser beam 118 via any of a variety of lasing mechanisms. For example, interrogation laser source 502 can comprise diode lasers. The interrogation laser beam 118 can be directed to beam splitter 504, which directs a portion of the interrogation laser beam 118 to reference detector 506 and the other portion of interrogation laser beam 118 to optics 508.

The optics 508 can comprise any of a variety of optical component arranged to condition and direct the interrogation laser beam 118 from beam splitter 504 to optical fiber 104 and direct the target reflected light 120 from optical fiber 104 to beam splitter 504. For example, the optics 508 can include polarizers, beam combiners, collimators, circulators, lenses, etc.

The beam splitter 504 may further be configured to direct the target reflected light 120 to the signal detector 510. The beam splitter 504 may include any of a variety of optical components used to direct incident light from the interrogation laser source 502 to the optics 508 and to direct incident light from the optics 508 to the signal detector 510.

The reference detector 506 and the signal detector 510 can be any of a variety of light detectors configured to measure an intensity and/or wavelength of the interrogation laser beam 118 and the target reflected light 120, respectively, and to output electrical signals based on the measured intensity and/or wavelength. For example, the reference detector 506 and/or the signal detector 510 can be a photomultiplier tube, a photodiode, or avalanche photodiodes. Electrical signals output from the reference detector 506 and the signal detector 510 can be communicated to a computing device (e.g., the computing sub-system 114, or the like) and used to determine the distance 126 between the distal end 124 of the optical fiber 104 and the target 108 as outlined above.

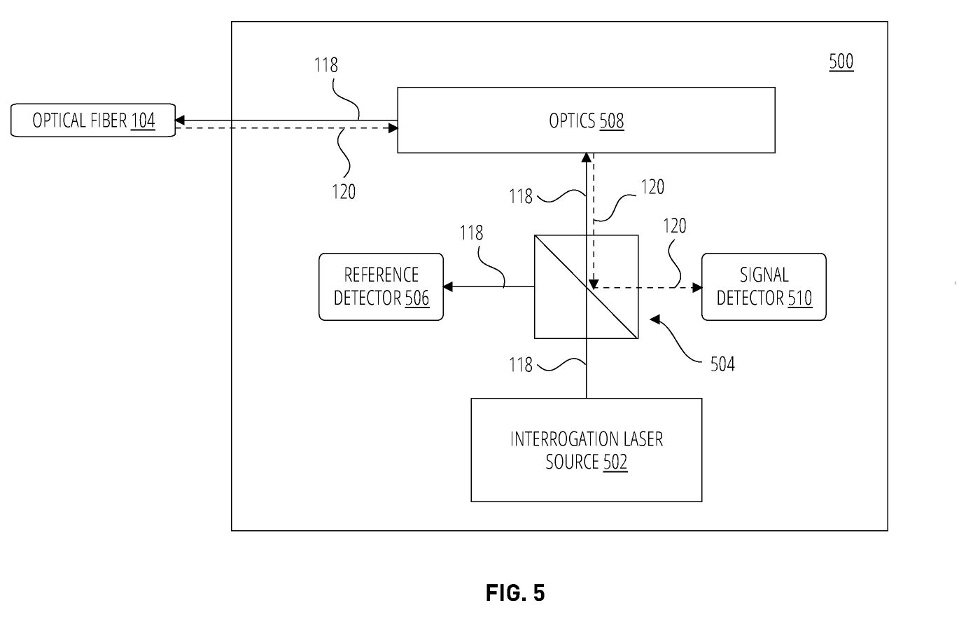

FIG. 6 illustrates a method 600 for providing a visual indication of a distance between an optical fiber and a target, according to at least one embodiment of the present disclosure. The method 600 can be implemented by a laser emitting medical device, such as, for example, the surgical laser system 100 of FIGS. 1A and 1B, the surgical laser system 200 of FIG. 2, or the surgical laser system 400 of FIG. 4. The method 600 will be described with reference to the surgical laser system 100 of FIGS. 1A and 1B as well as to the interrogation sub-system 500 for clarity of presentation. However, it is noted that method 600 could also be implemented by a laser emitting medical devices different from those described herein.

The method 600 can begin at block 602. At block 602 “determine a distance between a distal end of an optical fiber and a target” a distance between a distal end of an optical fiber and a target can be determined. For example, the distance 126 between the distal end 124 of the optical fiber 104 and the target 108 can be determined. In some embodiments, processing unit 128 can execute instructions 134 to: generate control signals for the interrogation laser source 502, which control signals when communicated to the interrogation laser source 502 cause the interrogation laser source 502 to generate interrogation laser beam 118; receive electrical signals from the reference detector 506 and signal detector 510 comprising indications of an intensity of the interrogation laser beam 118 and the target reflected light 120; and determine the distance 126 based on the electrical signals (or rather, the intensity of the interrogation laser beam 118 and the target reflected light 120).

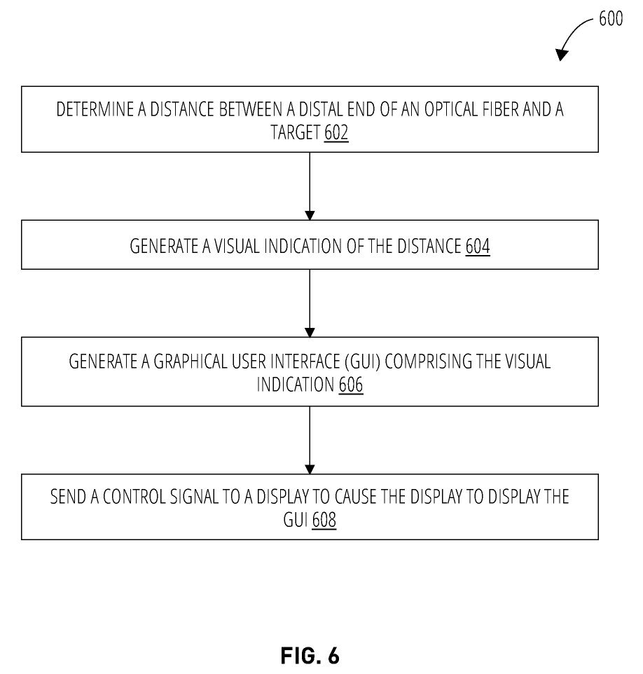

Continuing to block 604 “generate a visual indication of the distance” a visual indication of the distance can be generated. For example, processing unit 128 can execute instructions 134 to generate visual indication 136 based on the determined distance 126. Examples of visual indication 136 are provided below (e.g., with respect to FIGS. 7A - 7D). However, in general, visual indication 136 can comprise a quantitative visual representation of the distance. For example, with some embodiments, the visual representation can be numbers depicting the distance 126 in a common measurement (e.g., millimeters, inches, etc.) As another example, the visual representation can comprise words that describe the distance 126 (e.g., near, far, effective, non-effective, etc.) With yet other examples, the visual representation can comprise one or more geometric shapes where the geometric shape and/or characteristic of the geometric shape (e.g., color, size, fill, etc.) may change to indicate the distance. In yet other examples, the visual representation can comprise effects or animations, such as, fading or blinking, where the frequency of the fading and/or blinking may quantitatively indicate the distance. With still other examples, the visual representation can comprise symbols or known shapes (e.g., a check mark for within a desired distance, an exclamation mark for outside the desired distance, a stop sign, a stoplight arrangement, etc.). In yet other examples, combinations of the above examples can be used to visually represent the distance 126 (e.g., geometrical shapes with numerical distance values, or the like).

Continuing to block 606 “generate a graphical user interface (GUI) comprising the visual indication” a GUI comprising the visual indication can be generated. For example, processing unit 128 can execute instructions 134 to generate a graphical user interface for a surgical laser console (e.g., the surgical laser console 102, or the like), an endoscope console (e.g., the endoscope console 204, or the like) or another device as part of a surgical laser system (e.g., the display 402 of surgical laser system 400, or the like).

Continuing to block 608 “send a control signal to a display to cause the display to display the GUI” a control signal can be sent to a display to cause the display to display the GUI. For example, processing unit 128 can execute instructions 134 to send a control signal to a display (e.g., display 132, display 214, display 402, or the like) to cause the display to display the GUI, which comprises the visual indication 136. In such a manner, a visual indication of the distance 126 can be provided for a user (e.g., a physician, or the like) of the surgical laser system 100.

FIGS. 7A, 7B, 7C, and 7D, FIG. 7B, illustrate examples of visual indications of a distance that can be generated herein. In some embodiments, processing unit 128 can execute instructions 134 to generate visual indication 136 as depicted in FIGS. 7A - 7D. In particular, FIG. 7A illustrates an example visual indication 700a, which can comprise a filled circle 702 of one color (e.g., green, or the like) over a solid field 704 of another color (e.g., black, or the like) indicating a first distance or range of distances for the distance 126. In some embodiments, the filled circle 702 circle of visual indication 700a can be hollow (e.g., like depicted in FIGS. 7B and 7C.

FIG. 7B illustrates an example visual indication 700b, which can comprise hollow circle 706 of one color (e.g., yellow, or the like) over the solid field 704, indicating a second distance or range of distances for the distance 126. In some embodiments, the hollow circle 706 circle of visual indication 700b can be filled (e.g., like depicted in FIG. 7A).

FIG. 7C illustrates an example visual indication 700c, which can comprise a hollow circle 708 of one color (e.g., grey, or the like) over the solid field 704, indicating a third distance or range of distances for the distance 126. In some embodiments, the hollow circle 708 of visual indication 700c can be filled (e.g., like depicted in FIG. 7A). In some embodiments (e.g., where the hollow circle 706 and hollow circle 708 are hollow as depicted in these figures), the hollow circle 706 and hollow circle 708 can have a line width that is the same or different. For example, the line width of the hollow circle 706 in visual indication 700b is depicted as being heavier (or larger) than the line width of the hollow circle 708 in visual indication 700c.

FIG. 7D illustrates an example visual indication 700d, which can comprise a filled circle 710 of one color (e.g., orange, or the like) over the solid field 704 with a punctuation mark 712 (e.g., an exclamation mark, or the like) disposed within the filled circle 710, indicating an error condition and/or no distance measured. In some embodiments, the filled circle 710 of visual indication 700d can be hollow (e.g., like depicted in FIGS. 7B and 7C). With some embodiments, the filled circle 702, the hollow circle 706, the hollow circle 708, and/or the filled circle 710 can have the same diameters while in other embodiments the diameters of some or all of the circles can be different.

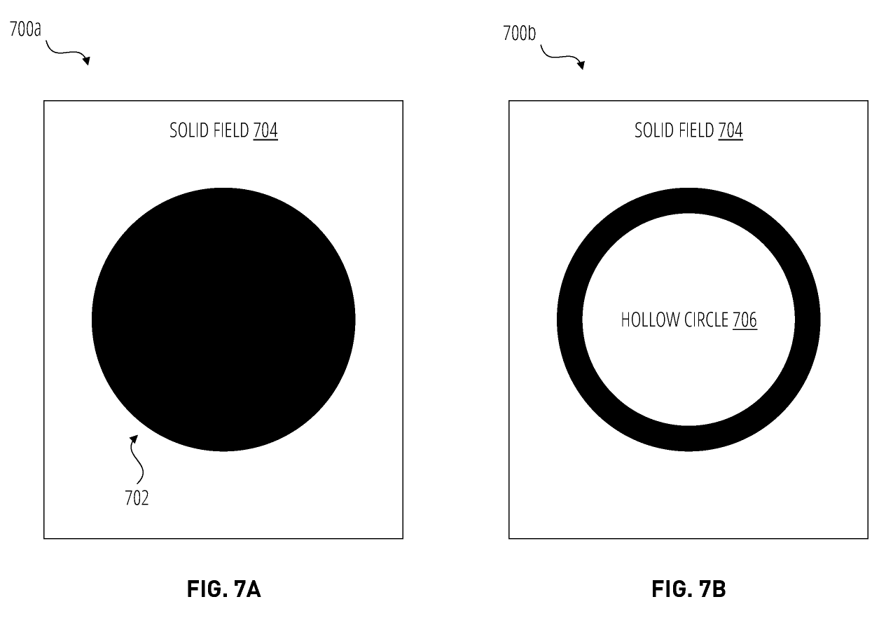

FIG. 8 illustrates a method 800 for providing a visual indication of a distance between an optical fiber and a target, according to at least one embodiment of the present disclosure. The method 800 can be implemented by a laser emitting medical device, such as, for example, the surgical laser system 100 of FIG. 1A and FIG. 1B, the surgical laser system 200 of FIG. 2, or the surgical laser system 400 of FIG. 4. The method 800 will be described with reference to the surgical laser system 100 of FIGS. 1A and 1B as well as to the interrogation sub-system 500 for clarity of presentation. Further, the method 800 is described with respect to the visual indications 700a to 700d of FIGS. 7A - 7D. However, it is noted that method 800 could also be implemented by a laser emitting medical devices different from those described herein and could generate visual indications different than those depicted herein.

The method 800 can begin at block 802. At block 802 “determine a distance between a distal end of an optical fiber and a target” a distance between a distal end of an optical fiber and a target can be determined. For example, the distance 126 between the distal end 124 of the optical fiber 104 and the target 108 can be determined. In some embodiments, processing unit 128 can execute instructions 134 to: generate control signals for the interrogation laser source 502, which control signals when communicated to the interrogation laser source 502 cause the interrogation laser source 502 to generate interrogation laser beam 118; receive electrical signals from the reference detector 506 and signal detector 510 comprising indications of an intensity of the interrogation laser beam 118 and the target reflected light 120; and determine the distance 126 based on the electrical signals (or rather, the intensity of the interrogation laser beam 118 and the target reflected light 120).

Continuing to decision block 804 “distance a real value?” a determination can be made as to whether the distance 126 is a real value. For example, processing unit 128 can execute instructions 134 to cause computing sub-system 114 to determine whether the distance 126 is a real value. From decision block 804, method 800 can continue to either block 806 or decision block 808. For example, method 800 can continue from decision block 804 to block 806 based on a determination that the distance is not a real value while method 800 can continue from decision block 804 to decision block 808 based on a determination that the distance 126 is a real value.

At block 806 “generate a visual indication indicating an error with the distance measurement system” a visual indication indicating that the distance is not a real value and/or no distance is measured can be generated. For example, processing unit 128 can execute instructions 134 to cause computing sub-system 114 to generate visual indication 136 like visual indication 700d of FIG. 7D.

At decision block 808 “distance less than a first threshold value?” a determination can be made of whether the distance is less than a first threshold value. For example, processing unit 128 can execute instructions 134 to cause computing sub-system 114 to determine whether the distance 126 is less than a first threshold value. In some embodiments, the first threshold value can be configurable by a user (e.g., a physician, or the like). In some embodiments, the first threshold can be 1.3 millimeters (mm). In some embodiments, the first threshold can be 1.5 mm, in some embodiments, the first threshold can be between 1.0 and 2.0 mm. From decision block 808, method 800 can continue to either block 810 or decision block 812. For example, method 800 can continue from decision block 808 to block 810 based on a determination that the distance is less than the first threshold value while method 800 can continue from decision block 808 to decision block 812 based on a determination that the distance 126 is not less than the first threshold value.

At block 810 “generate a visual indication indicating the distance is less than the first threshold value” a visual indication indicating that the distance is less than the first threshold value can be generated. For example, processing unit 128 can execute instructions 134 to cause computing sub-system 114 to generate visual indication 136 like visual indication 700a of FIG. 7A.

At decision block 812 “distance less than a second threshold value?” a determination can be made of whether the distance is less than a second threshold value. For example, processing unit 128 can execute instructions 134 to cause computing sub-system 114 to determine whether the distance 126 is less than a second threshold value. In some embodiments, the second threshold value can be configurable by a user (e.g., a physician, or the like). In some embodiments, the second threshold can be 2.5 mm. In some embodiments, the second threshold can be 3.0 mm, in some embodiments, the second threshold can be between 2.0 mm and 4.0 mm. From decision block 812, method 800 can continue to either block 814 or block 816. For example, method 800 can continue from decision block 812 to block 814 based on a determination that the distance is less than the second threshold value while method 800 can continue from decision block 812 to block 816 based on a determination that the distance 126 is not less than the second threshold value.

At block 814 “generate a visual indication indicating the distance is greater than or equal to the first threshold value but less than the second threshold value” a visual indication indicating that the distance is greater than or equal to the first threshold value but less than the second threshold value can be generated. For example, processing unit 128 can execute instructions 134 to cause computing sub-system 114 to generate visual indication 136 like visual indication 700b of FIG. 7B.

At block 816 “generate a visual indication indicating the distance is greater than or equal to the second threshold value” a visual indication indicating that the distance is greater than or equal to the second threshold value can be generated. For example, processing unit 128 can execute instructions 134 to cause computing sub-system 114 to generate visual indication 136 like visual indication 700c of FIG. 7C.

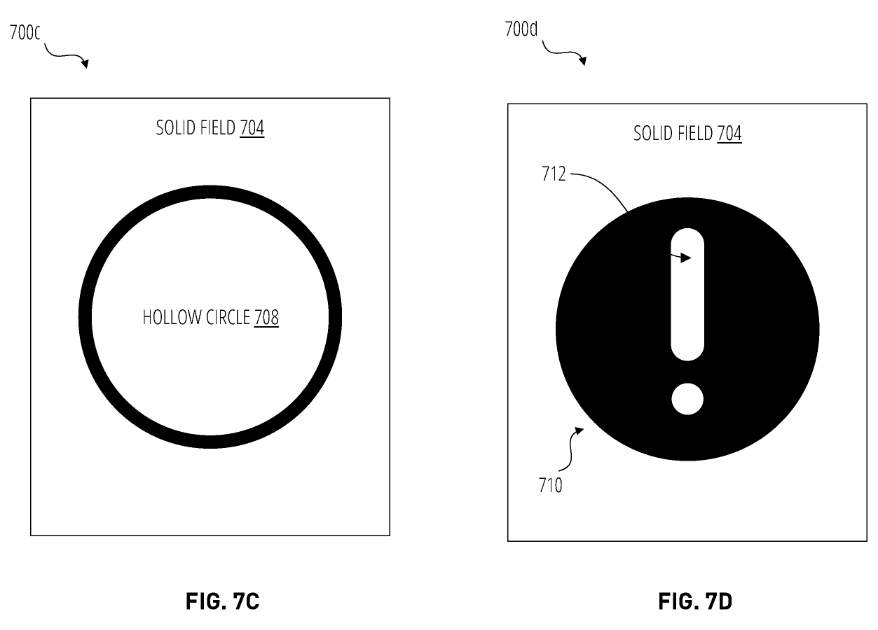

FIGS. 9 - 12 illustrate example GUIs comprising a visual indication of a distance, which can be generated according to at least one embodiment of the disclosure. Turning specifically to FIG. 9, laser console GUI 900 is depicted displayed on display 132. In some examples, processing unit 128 can execute instructions 134 to cause computing sub-system 114 to generate laser console GUI 900 and display laser console GUI 900 on display 132. Laser console GUI 900 can comprise surgical laser console settings icons 902a and/or 902b as well as visual indication 136. In some embodiments, visual indication 136 can be generated based on method 800 and can correspond to the visual indications 700a, 700b, 700c, or 700d depicted in FIGS. 7A - 7D.

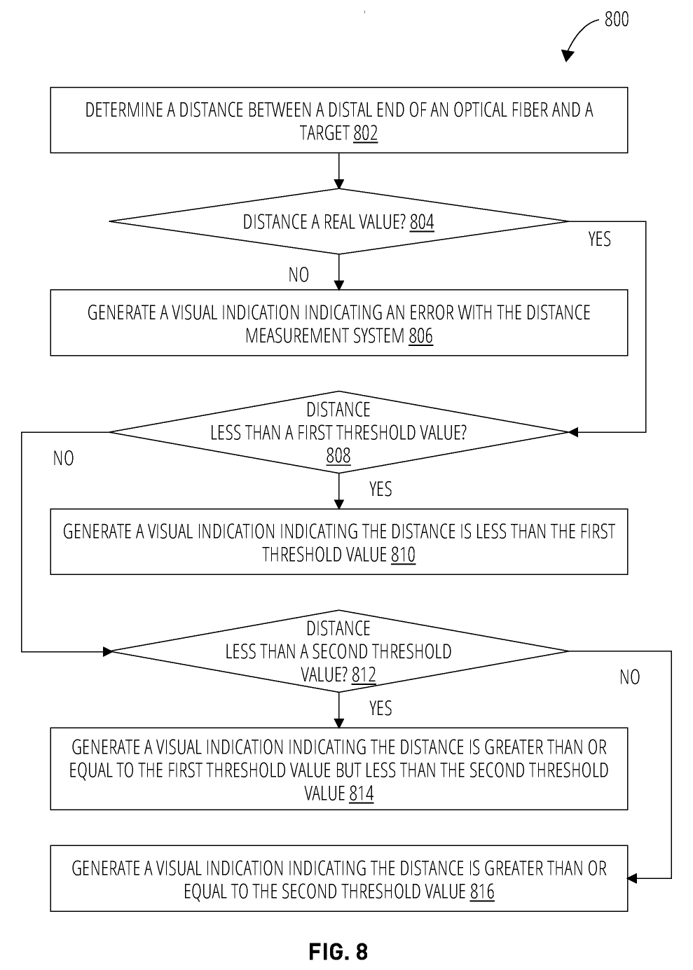

FIG. 10 depicts endoscope GUI 1000 is depicted displayed on display 214. In some examples, processing unit 128 can execute instructions 134 to cause computing sub-system 114 to generate visual indication 136 based on method 800 where the visual indication 136 can correspond to the visual indications 700a, 700b, 700c, or 700d depicted in FIGS. 7A - 7D. Further, processing unit 128 can execute instructions 134 to cause computing sub-system 114 to send indications of visual indication 136 to computing sub-system 208. Additionally, processing unit 210 can execute instructions 216 to generate endoscope GUI 1000 comprising the visual indication 136 and display endoscope GUI 1000 on display 214. Endoscope GUI 1000 can comprise an endoscope image 1002, endoscope settings icons 1004, as well as visual indication 136.

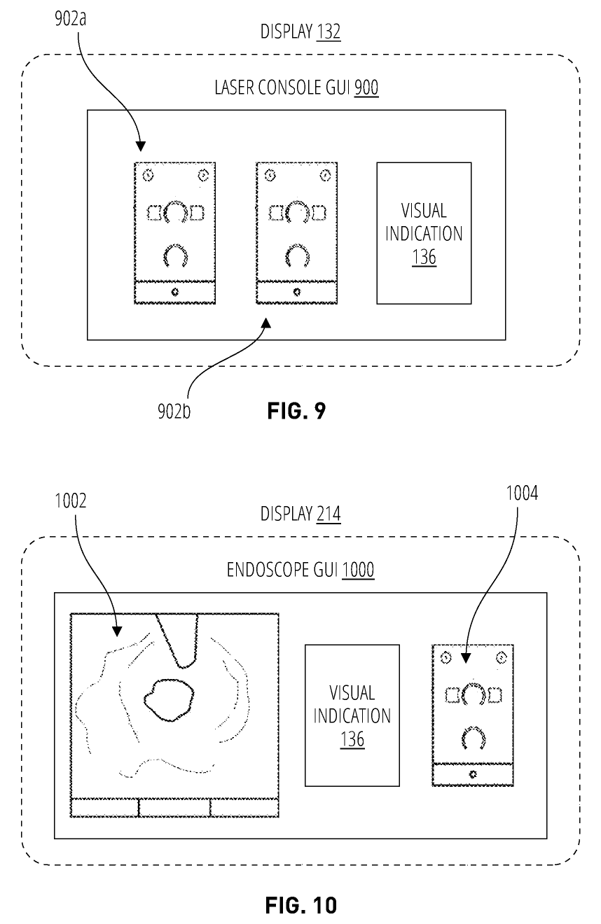

FIG. 11 depicts endoscope GUI 1100, which can be like endoscope GUI 1000 for an endoscope console and/or a multi-system viewer. For example, the display 214 or the display 312 can be configured to display endoscope GUI 1100.

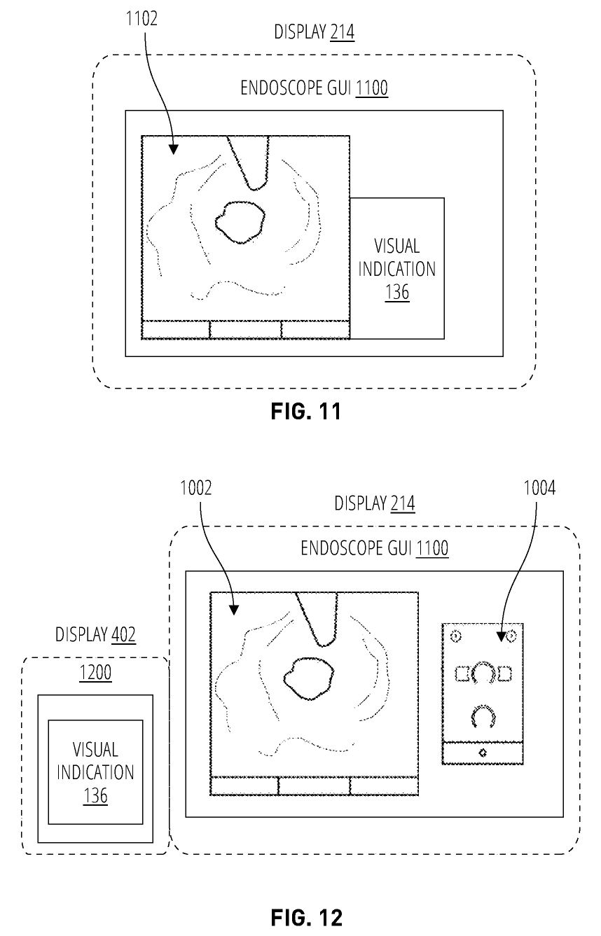

FIG. 12 depicts external display GUI 1200 is depicted displayed on display 402. In some examples, display 402 can be attached to a surgical laser console (e.g., surgical laser console 102, or the like) or an endoscope display (e.g., display 214, or the like). Processing unit 128 can execute instructions 134 to cause computing sub-system 114 to generate visual indication 136 based on method 800 where the visual indication 136 can correspond to the visual indications 700a, 700b, 700c, or 700d depicted in FIGS. 7A - 7D. Further, processing unit 128 can execute instructions 134 to generate external display GUI 1200 including the visual indication 136 and cause the display 402 to display the external display GUI 1200.

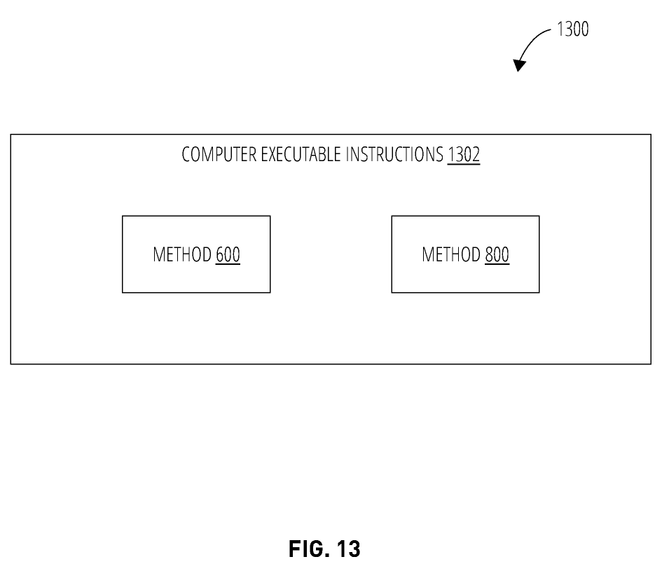

FIG. 13 illustrates computer-readable storage medium 1300. Computer-readable storage medium 1300 may comprise any non-transitory computer-readable storage medium or machine-readable storage medium, such as an optical, magnetic or semiconductor storage medium. In various embodiments, computer-readable storage medium 1300 may comprise an article of manufacture. In some embodiments, computer-readable storage medium 1300 may store computer executable instructions 1302 with which circuitry (e.g., computing sub-system 114, processing unit 128, or the like) can execute. For example, computer executable instructions 1302 can include instructions to implement operations described with respect to method 600, and/or method 800. Examples of computer-readable storage medium 1300 or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions 1302 may include any suitable type of code, such as source code, compiled code, interpreted code, executable code, static code, dynamic code, object-oriented code, visual code, and the like.

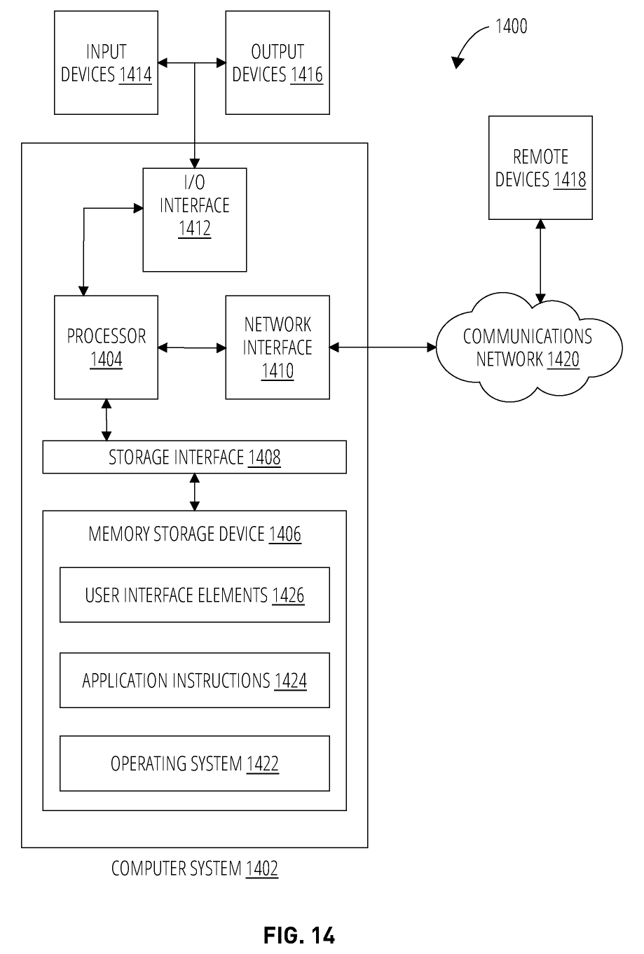

FIG. 14 is a block diagram of a computing environment 1400 including a computer system 1402 for implementing embodiments consistent with the present disclosure. In some embodiments, the computing environment 1400, or portion thereof (e.g., the computer system 1402) may comprise or be comprised in a laser system (e.g., the computing sub-system 114 of the surgical laser system 100 can embody portions of the computing environment 1400. Accordingly, in various embodiments, computer system 1402 may determine a distance between a distal end of the optical fiber and a target and generate visual indications and/or GUIs indicating the distance during a surgical laser procedure (e.g., laser lithotripsy, or the like).

The computer system 1402 may include a central processing unit (“CPU” or “processor”) 1404. The processor 1404 may include at least one data processor for executing instructions and/or program components for executing user or system-generated processes. A user may include a person, a person using a device such as those included in this disclosure, or another device. The processor 1404 may include specialized processing units such as integrated system (bus) controllers, memory management control units, floating point units, graphics processing units, neural processing units, digital signal processing units, etc. The processor 1404 may be disposed in communication with input devices 1414 and output devices 1416 via I/O interface 1412. The I/O interface 1412 may employ various signaling (or communication) protocols and can include analog and/or digital signaling protocols. Further, the I/O interface 1412 can include wired and/or wireless signaling protocols. As an example, the I/O interface signaling protocols can include mono, stereo, serial bus, parallel bus, coaxial, component, composite, radio frequency (RF), cellular, or the like.

Using the I/O interface 1412, computer system 1402 may communicate with input devices 1414 and output devices 1416. In some embodiments, the processor 1404 may be disposed in communication with a communications network 1420 via a network interface 1410. In various embodiments, the communications network 1420 may be utilized to communicate with a remote memory storage device 1406, such as for accessing look-up tables, performing updates, or utilizing external resources. The network interface 1410 may communicate with the communications network 1420. The network interface 1410 may employ connection protocols including, without limitation, direct connect, wired and/or wireless Ethernet, or the like.

The communications network 1420 can be implemented as one of the different types of networks, such as intranet or Local Area Network (LAN), Closed Area Network (CAN) and such. The communications network 826 may either be a dedicated network or a shared network, which represents an association of the different types of networks that use a variety of protocols, for example, Hypertext Transfer Protocol (HTTP), CAN Protocol, Transmission Control Protocol/Internet Protocol (TCP/IP), Wireless Application Protocol (WAP), etc., to communicate with each other. Further, the communications network 1420 may include a variety of network devices, including routers, bridges, servers, computing devices, storage devices, etcetera. In some embodiments, the processor 1404 may be disposed in communication with a memory storage device 1406 via a storage interface 1408. The storage interface 1408 may connect to memory storage device 1406 including, without limitation, memory drives, removable disc drives, etc., employing connection protocols such as Serial Advanced Technology Attachment (SATA), Integrated Drive Electronics (IDE), Universal Serial Bus (USB), fiber channel, Small Computer Systems Interface (SCSI), or the like. The memory drives may further include a drum, magnetic media, optical media, solid-state circuitry, or some combination of the above.

Furthermore, memory storage device 1406 may include one or more computer-readable storage media utilized in implementing embodiments consistent with the present disclosure. Generally, a computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., non-transitory. Examples include Random Access Memory (RAM), Read-Only Memory (ROM), non-volatile memory, hard drives, Compact Disc (CD) ROMs, Digital Video Disc (DVDs), flash drives, disks, and any other known physical storage media.

The memory storage device 1406 may store a collection of program or database components, including, without limitation, an operating system 1422, application instructions 1424, and user interface elements 1426. In various embodiments, the operating system 1422 may facilitate resource management and operation of the computer system 1402. Examples of operating systems include, without limitation, UNIX^{®} and/or UNIX-like distributions (e.g., Berkely Software Distribution^{®} (BSD), FreeBSD^{®}, NetBSD^{®}, OpenBSD^{®}, etc.), Linux^{®} distributions (e.g., Red Hat^{®}, Ubuntu^{®}, etc.), IBM^{®}OS/2^{®}, Microsoft®, iOS®, macOS®, or the like.

The application instructions 1424 may include instructions that when executed by the processor 1404 cause the processor 1404 to perform one or more techniques, steps, procedures, and/or methods described herein, such as to generate visual indications 136 and/or GUIs (e.g., laser console GUI 900, endoscope GUI 1000, endoscope GUI 1100, external display GUI 1200, etc.)

The user interface elements 1426 may facilitate display, execution, interaction, manipulation, or operation of program components through textual or graphical facilities. For example, user interfaces may provide computer interaction interface elements on a display system operatively connected to the computer system 1402, such as cursors, icons, checkboxes, menus, scrollers, windows, widgets, etcetera. The user interface elements 1426 may be employed by application instructions 1424 and/or operating system 1422 to provide, for example, a user interface with which a user can interact with computer system 1402. In some embodiments, the user interface elements 1426 may be displayed on a display.

Terms used herein should be accorded their ordinary meaning in the relevant arts, or the meaning indicated by their use in context, but if an express definition is provided, that meaning controls.

Herein, references to "one embodiment" or "an embodiment" do not necessarily refer to the same embodiment, although they may. Unless the context clearly requires otherwise, throughout the description and the claims, the words "comprise," "comprising," and the like are to be construed in an inclusive sense as opposed to an exclusive or exhaustive sense; that is to say, in the sense of "including, but not limited to." Words using the singular or plural number also include the plural or singular number respectively, unless expressly limited to one or multiple ones. Additionally, the words "herein," "above," "below" and words of similar import, when used in this application, refer to this application as a whole and not to any portions of this application. When the claims use the word "or" in reference to a list of two or more items, that word covers all the following interpretations of the word: any of the items in the list, all the items in the list and any combination of the items in the list, unless expressly limited to one or the other. Any terms not expressly defined herein have their conventional meaning as commonly understood by those having skill in the relevant art(s).

What is claimed is:

1. A multi-system viewer, comprising:
a processor;
a display coupled to the processor; and
memory coupled to the processor and comprising instructions, which instructions when executed by the processor cause the multi-system viewer to:
generate, during a surgical procedure, a visual representation of a distance between a distal end of an optical fiber and a target, and
send, during the surgical procedure, control signals to the display to cause the display to display the visual representation,
wherein the target is disposed in a treatment environment and wherein the optical fiber is inserted into the treatment environment via a working channel of an endoscope.

2. The multi-system viewer of claim 16 the instructions when executed by the processor further cause the multi-system viewer to receive, during the surgical procedure, an indication of the distance from a surgical laser console.

3. The multi-system viewer of claim 1, the instructions when executed by the processor further cause the multi-system viewer to receive, during the surgical procedure, an indication of the distance from an endoscope console.

4. 19 The multi-system viewer of claim 3, the instructions when executed by the processor further cause the multi-system viewer to receive, during the surgical procedure, indications of an image of the target.

5. The multi-system viewer of claim 4, the instructions when executed by the processor further cause the multi-system viewer to send the control signals to the display to cause the display to display both the visual representation and the image of the target.

6. The multi-system viewer of claim 1, the instructions when executed by the processor further cause the multi-system viewer to generate a graphical user interface (GUI) comprising at least the visual representation, wherein the control signals cause the display to display the GUI during the surgical procedure.

7. The multi-system viewer of claim 1, wherein the visual representation comprises one or more geometric shapes, one or more numbers, and/or one or more words.

8. The multi-system viewer of claim 1, the instructions when executed by the processor further cause the multi-system viewer to:
determine, during the surgical procedure, whether the distance is less than a threshold value,
wherein the visual representation is a first visual representation comprising a quantitative indication that the distance is less than the threshold value based on a determination that the distance is less than the threshold value, or
wherein the visual representation is a second visual representation comprising a quantitative indication that the distance is greater than or equal to the threshold value based on a determination that the distance is not less than the threshold value.

9. The multi-system viewer of claim 8, wherein the first visual representation comprises a solid circle and wherein the second visual representation comprises a hollow circle.

10. An endoscope console, comprising
a processor;
a network interconnect coupled to the processor;
a display coupled to the processor; and
memory coupled to the processor and comprising instructions, which when executed by the processor cause the endoscope console to:
receive, from a surgical laser console via the network interconnect during a surgical procedure, an indication of a distance between a distal end of an optical fiber and a target,
generate, during the surgical procedure, a visual representation of the distance, and
send, during the surgical procedure, control signals to the display to cause the display to display the visual representation,
wherein the target is disposed in a treatment environment and wherein the optical fiber is inserted into the treatment environment via a working channel of an endoscope.

11. The endoscope console of claim 10, the instructions when executed by the processor further cause the endoscope console to receive, during the surgical procedure, indications of an image of the target.

12. The endoscope console of claim 11, the instructions when executed by the processor further cause the endoscope console to send the control signals to the display to cause the display to display both the visual representation and the image of the target.

13. The endoscope console of claim 10, wherein the visual representation comprises one or more geometric shapes, one or more numbers, and/or one or more words.

14. The endoscope console of claim 10, the instructions when executed by the processor further cause the endoscope console to:
determine, during the surgical procedure, whether the distance is less than a threshold value,
wherein the visual representation is a first visual representation comprising a quantitative indication that the distance is less than the threshold value based on a determination that the distance is less than the threshold value, or
wherein the visual representation is a second visual representation comprising a quantitative indication that the distance is greater than or equal to the threshold value based on a determination that the distance is not less than the threshold value.

15. The endoscope console of claim 14, wherein the first visual representation comprises a solid circle and wherein the second visual representation comprises a hollow circle.

16. A surgical laser console, comprising
a network interconnect;
a processor; and
memory coupled to the processor and comprising instructions, which when executed by the processor cause the surgical laser console to:
determine, during a surgical procedure, a distance between a distal end of an optical fiber and a target,
generate, during the surgical procedure, a visual representation of the distance, and
send, to an endoscope console via the network interconnect during the surgical procedure, an indication of the visual representation and control signals to cause a display of the endoscope console to display the visual representation as part of a graphical user interface (GUI),
wherein the target is disposed in a treatment environment and wherein the optical fiber is inserted into the treatment environment via a working channel of an endoscope.

17. The surgical laser console of claim 16, wherein the GUI comprises both the visual representation and an image of the target.

18. The surgical laser console of claim 16, wherein the visual representation comprises one or more geometric shapes, one or more numbers, and/or one or more words.

19. The surgical laser console of claim 16, the instructions when executed by the processor further cause the surgical laser console to:
determine, during the surgical procedure, whether the distance is less than a threshold value,
wherein the visual representation is a first visual representation comprising a quantitative indication that the distance is less than the threshold value based on a determination that the distance is less than the threshold value, or
wherein the visual representation is a second visual representation comprising a quantitative indication that the distance is greater than or equal to the threshold value based on a determination that the distance is not less than the threshold value.

20. The surgical laser console of claim 34, wherein the first visual representation comprises a solid circle and wherein the second visual representation comprises a hollow circle.
